(19) United States
(12) Patent Application Publication
(10) Pub. No.: US 2025/0145568 A1
(43) Pub. Date: May 8, 2025

(54) DEUTERATED ORGANIC COMPOUNDS AND USES THEREOF

(71) Applicant: ENGRAIL THERAPEUTICS, INC., San Diego, CA (US)

(72) Inventors: Krishna VADODARIA, San Diego, CA (US); Kimberly VANOVER, Tulsa, OK (US); Vikram SUDARSAN, San Diego, CA (US); David GARVEY, Dover, MA (US)

(21) Appl. No.: 18/761,811

(22) Filed: Jul. 2, 2024

Related U.S. Application Data
(60) Provisional application No. 63/511,685, filed on Jul. 2, 2023.
(60) Provisional application No. 63/511,847, filed on Jul. 3, 2023.
(60) Provisional application No. 63/511,849, filed on Jul. 3, 2023.
(60) Provisional application No. 63/511,852, filed on Jul. 3, 2023.
(60) Provisional application No. 63/511,853, filed on Jul. 3, 2023.
(60) Provisional application No. 63/511,855, filed on Jul. 3, 2023.
(60) Provisional application No. 63/512,064, filed on Jul. 5, 2023.

(51) Int. Cl. C07D 207/09; A61K 31/402; A61P 25/22; C07B 59/00

(52) U.S. Cl. CPC C07D 207/09; A61K 31/402; A61P 25/22; C07B 59/004; C07B 2200/05

(57) ABSTRACT

Provided are compounds of Formula I, described herein, processes for their preparation, their use as pharmaceuticals, and pharmaceutical compositions comprising them and intermediates used in their preparation. Compounds of Formula I are useful, for instance, in modulating dopamine neurotransmission and treating disorders that may benefit from the same, such as schizophrenia and depression.

This application claims priority to U.S. Provisional Application No. 63/511,685 filed Jul. 2, 2023, U.S. Provisional Application No. 63/511,847 filed Jul. 3, 2023, U.S. Provisional Application No. 63/511,849 filed Jul. 3, 2023, U.S. Provisional Application No. 63/511,852 filed Jul. 3, 2023, U.S. Provisional Application No. 63/511,853 filed Jul. 3, 2023, U.S. Provisional Application No. 63/511,855 filed Jul. 3, 2023, and U.S. Provisional Application No. 63/512,064 filed Jul. 5, 2023, the contents of each of which are hereby incorporated by reference in their entireties.

### FIELD

Provided are compounds of Formula I, described below, processes for their preparation, their use as pharmaceuticals, and pharmaceutical compositions comprising them and intermediates used in their preparation. Compounds of Formula I are useful, for instance, in modulating dopamine neurotransmission and treating disorders that may benefit from the same, such as schizophrenia and depression.

### BACKGROUND

Dopamine is involved in a variety of central nervous system functions, including voluntary movement, feeding, affect, reward, sleep, attention, working memory, and learning. Dopaminergic dysfunction can lead to diseases such as schizophrenia and depression.

When released from presynaptic terminals, dopamine activates members of a family of G protein-coupled dopamine receptors D1-D5. Dopamine receptors (D1-D5) are divided into two groups, the D1-like (D1 and D5) and the D2-like (D2, D3, and D4). Activation of D1-like receptors activates adenylyl cyclase and increases cAMP levels. D2-like receptors are inhibitory. Activation of D2-like receptors inhibits activation of adenylyl cyclase.

D1-like receptors are found postsynaptically on dopamine-receptive cells, while D2-like dopamine receptors are expressed both postsynaptically on dopamine target cells and presynaptically on dopaminergic neurons.

Antipsychotics are used to manage psychosis, in particular schizophrenia. A hallmark of antipsychotics is D2 receptor antagonism. D2 receptor antagonism is effective in reducing positive symptoms of schizophrenia (for instance, hallucinations and delusions), but often also produces extrapyramidal side effects, including parkinsonism, akathisia, and tardive dyskinesia, increases prolactin, and may exacerbate negative symptoms of schizophrenia (for instance, loss of interest and motivation in life and activities, social withdrawal, and anhedonia). Many psychotic patients also suffer from depression, which may be left untreated by current medications.

In addition to effects on dopamine receptors, such as D2, D3, and D4, antipsychotics may also have effects on serotonin receptors, such as 5-HT1A, 5-HT2A, 5-HT2C, 5-HT6, and 5-HT7. Interacting with dopamine and serotonin receptors may be beneficial, for instance, resulting in reduced extrapyramidal motor side effects (EPS) liability. However, multi-target drugs may also result in undesirable off-target side effects.

Because imbalances in dopamine can lead to a variety of disorders and current medications may not be able to effectively modulate dopamine levels and may have undesirable side effects, new compounds that can modulate dopamine neurotransmission are needed, as are methods of treating diseases that involve imbalances in dopamine.

### BRIEF SUMMARY

Provided is a compound of Formula X:

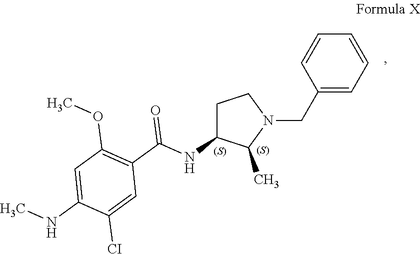

wherein six or more hydrogens are replaced by deuterium (i.e., six or more hydrogen positions have a significantly greater than natural abundance of deuterium at that position), in free or pharmaceutically acceptable salt form.

Further provided are pharmaceutical compositions comprising compounds of Formula X, processes for preparing compounds of Formula X, and pharmaceutical uses of compounds of Formula X, for instance, as an anti-anhedonic agent and to treat schizophrenia and depression.

For instance, provided is a compound of Formula I:

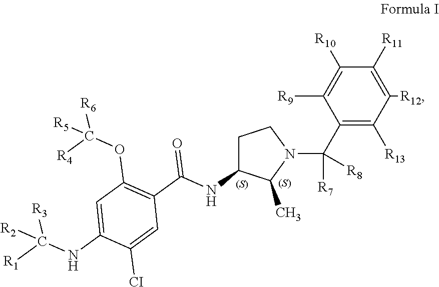

wherein:
R₁, R₂, R₃, R₄, R₅, R₆, R₇, R₈, R₉, R₁₀, R₁₁, R₁₂, and R₁₃ are independently selected from H and D;
and wherein
at least one of R₁, R₂, and R₃, is D;
and at least one of R₉, R₁₀, R₁₁, R₁₂, and R₁₃ is D;
in free or salt form.

Further provided are pharmaceutical compositions comprising compounds of Formula I, processes for preparing compounds of Formula I, and pharmaceutical uses of compounds of Formula I, for instance, as an anti-anhedonic agent and to treat schizophrenia and depression.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

In the event of a conflict in a definition in the present disclosure and that of a cited reference, the present disclosure controls.

D2- and D3-receptors are expressed both postsynaptically on dopamine target cells and presynaptically on dopamine neurons. Dopamine receptors are mainly located on non-dopamine neurons. Dopamine receptors on dopamine neurons are called autoreceptors. Autoreceptors contribute to regulating dopamine neuron activity and controlling the synthesis, release, and uptake of dopamine.

Presynaptic D2-like dopamine autoreceptors regulate dopamine release. A low dose of a D2-like receptor antagonist may preferentially block presynaptic autoreceptors and increase dopamine release, while a high dose may block postsynaptic receptors and decrease dopamine neurotransmission. Relatively high occupancy of D2-like receptors has been associated with antipsychotic effects, while lower occupancy has been associated with antidepressant effects.

Anhedonia is a core symptom of major depressive disorder (MDD) and is associated with inadequate response to approved selective serotonin reuptake inhibitors (SSRIs) and serotonin norepinephrine reuptake inhibitors (SNRIs) and psychotherapy (e.g., cognitive behavioral therapy (CBT)) and neurostimulation (e.g., transcranial magnetic stimulation (TMS)). There remains a need for effective treatment of MDD characterized by anhedonia. Despite a range of available therapies, up to 50% of people suffering from MDD fail to respond to treatments, and only about 30% of patients fully recover after receiving currently available antidepressants and treatment outcomes are even poorer for MDD individuals with anhedonia.

Depletion of dopamine/catecholamines induces symptoms of depression and anhedonia. Increasing dopamine neurotransmission can alleviate symptoms of depression and anhedonia. However, while a high dose of a dopamine D2/D3 agonist may activate dopamine post-synaptic receptors, it can also be poorly tolerated (e.g., nausea/vomiting). Low dose of a dopamine D2/D3 receptor antagonist may preferentially block pre-synaptic dopamine autoreceptors and increase dopamine release without being poorly tolerated.

Besides MDD, anhedonia also plays a role in bipolar disorder, schizophrenia, post-traumatic stress disorder, and substance use disorder. Despite its role in many disorders, there are no approved medications to treat anhedonia.

The IUPAC name of nemonapride is (+)-cis-N-(1-Benzyl-2-methylpyrrolidin-3-yl)-5-chloro-2-methoxy-4-methylaminobenzamide. Nemonapride is described in U.S. Pat. No. 4,210,660 as a strong central nervous system depressant, in particular a strong antipsychotic.

Nemonapride is a dopamine D2/D3/D4 receptor antagonist. Nemonapride is approved in Japan and South Korea for treatment of schizophrenia. Nemonapride is supplied as 3 mg and 10 mg tablets. The approved daily dose of nemonapride for schizophrenia is 9 to 36 mg given orally in divided doses after meals. The dose can be increased up to 60 mg daily.

The nemonapride prescribing information indicates that the elimination half-life when nemonapride 3 mg and 6 mg was administered orally to healthy adults was 2.3 to 4.5 hours. Urinary metabolites of nemonapride result from debenzylation and N-demethylation. See Emilace package insert.

In addition to being a dopamine D2/D3/D4 receptor antagonist, nemonapride is also a 5-HT1A agonist and has been reported to bind to 5-HT2A.

When a drug is used as a mixture of stereoisomers, it is not possible to predict what properties (e.g., biological target, pharmacokinetics) each stereoisomer has, especially a drug that has multiple biological targets.

Dopamine postsynaptic receptor antagonism reduces psychosis, particularly in schizophrenia, by reducing dopamine neurotransmission. A low dose of a dopamine receptor antagonist may selectively block presynaptic autoreceptors resulting in increased dopamine release and enhanced dopamine neurotransmission. Selective dopamine receptor antagonists limit off target interactions. Off target interactions may contribute to side effects in drugs that are not as selective.

Pharmacokinetics of deuterated compounds disclosed herein are beneficial, for instance, showing one or more of the following: enriched brain levels compared to plasma levels; brain:plasma exposure supporting once-daily dosing; and extended brain enrichment compared to plasma levels, which may allow for higher and more sustained receptor occupancy with less frequent dosing and may be associated with fewer peripheral side effects. Nemonapride is taken in multiple doses per day.

Compounds that are dopamine receptor antagonists modulate dopamine neurotransmission and are therefore useful in treating disorders involving dopamine signaling pathways, for instance, disorders involving D2, D3, and/or D4 receptors.

Provided is a compound of Formula X:

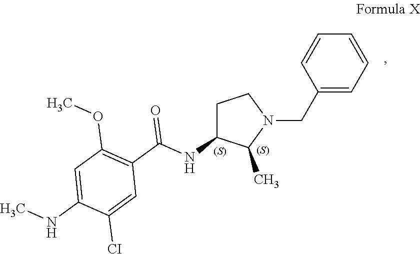

wherein six or more hydrogens are replaced by deuterium (i.e., six or more hydrogen positions have a significantly greater than natural abundance of deuterium at that position), in free or pharmaceutically acceptable salt form.

Further provided are pharmaceutical compositions comprising compounds of Formula X, processes for preparing compounds of Formula X, and pharmaceutical uses of compounds of Formula X, for instance, as an anti-anhedonic agent and to treat schizophrenia and depression.

For instance, provided is a compound of Formula I:

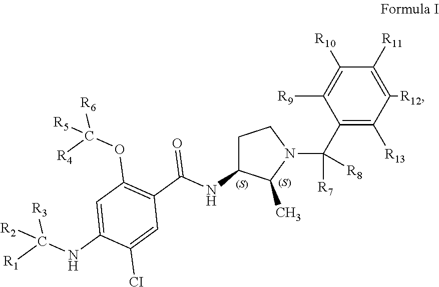

wherein:
R₁, R₂, R₃, R₄, R₅, R₆, R₇, R₈, R₉, R₁₀, R₁₁, R₁₂, and R₁₃ are independently selected from H and D;
and wherein
at least one of R₁, R₂, and R₃, is D;
and at least one of R₉, R₁₀, R₁₁, R₁₂, and R₁₃ is D;
in free or salt form.

Further provided are compounds of Formula I as follows:
1.1 Formula I, wherein the compound is in pharmaceutically acceptable salt form.
1.2 Formula I, wherein the compound is in free form.
1.3 Any of Formula I, 1.1, or 1.2, wherein each of R₁, R₂, and R₃ are D.
1.4 Any of Formula I or 1.1-1.3, wherein each of R₄, R₅, and R₆ are D.
1.5 Any of Formula I or 1.1-1.4, wherein R₇ and R₈ are D.
1.6 Any of Formula I or 1.1-1.5, wherein each of R₉, R₁₀, R₁₁, R₁₂, and R₁₃ are D.
1.7 Any of Formula I or 1.1-1.6, wherein each of R₁, R₂, R₃, R₄, R₅, R₆, R₇, R₈, R₉, R₁₀, R₁₁, R₁₂, and R₁₃ are D.
1.8 Any of Formula I or 1.1-1.7, wherein the compound is:

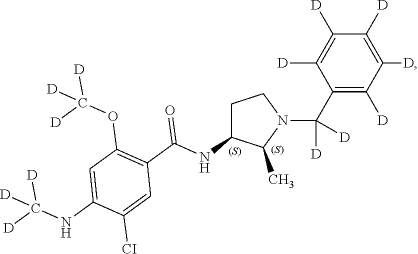

in free or salt form, e.g., in free or pharmaceutically acceptable salt form, e.g., in free form.
1.9 Any of Formula I or 1.1-1.8, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.10 Any of Formula I or 1.1-1.9, wherein the compound, in free or salt form (e.g., pharmaceutically acceptable salt form), has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Formula I or 1.1-1.9, wherein the compound, in free or salt form (e.g., pharmaceutically acceptable salt form), has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.11 Any of Formula I or 1.1-1.10, wherein the compound is substantially stereoisomerically pure. For instance, wherein the compound has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the compound is substantially diastereomerically and/or enantiomerically pure, e.g., wherein the compound is substantially diastereomerically and enantiomerically pure.
1.12 Any of Formula I or 1.1-1.11, wherein the compound is substantially diastereomerically pure. For instance, wherein the compound has a diastereomeric excess of greater than 90%, e.g., a diastereomeric excess equal to or greater than 95%, e.g., a diastereomeric excess equal to or greater than 96%, e.g., a diastereomeric excess equal to or greater than 97%, e.g., a diastereomeric excess equal to or greater than 98%, e.g., a diastereomeric excess equal to or greater than 99%.
1.13 Any of Formula I or 1.1-1.12, wherein the compound is substantially enantiomerically pure. For instance, wherein the compound has an enantiomeric excess of greater than 90%, e.g., an enantiomeric excess equal to or greater than 95%, e.g., an enantiomeric excess equal to or greater than 96%, e.g., an enantiomeric excess equal to or greater than 97%, e.g., an enantiomeric excess equal to or greater than 98%, e.g., an enantiomeric excess equal to or greater than 99%.
1.14 Any of Formula I or 1.1-1.13, wherein the compound has the stereochemical configuration as shown in Formula I.
1.15 Any of Formula I or 1.1-1.14, wherein the compound is in a pharmaceutical composition with a pharmaceutically acceptable carrier. For instance, any of Formula I or 1.1-1.14, wherein an effective amount of the compound is in a pharmaceutical composition with a pharmaceutically acceptable carrier.

Further provided is a pharmaceutical composition comprising a compound of Formula X, in free or pharmaceutically acceptable salt form, and a pharmaceutically acceptable carrier.

Further provided is a pharmaceutical composition (Composition 1) comprising a compound of Formula I (e.g., any of Formula 1.1-1.15):

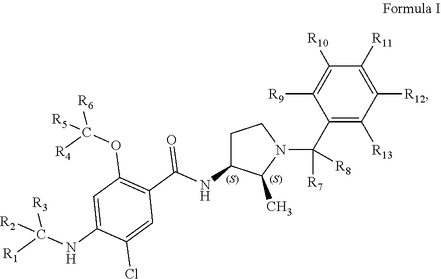

wherein:
R₁, R₂, R₃, R₄, R₅, R₆, R₇, R₈, R₉, R₁₀, R₁₁, R₁₂, and R₁₃ are independently selected from H and D;
and wherein
at least one of R₁, R₂, and R₃, is D;
and at least one of R₉, R₁₀, R₁₁, R₁₂, and R₁₃ is D;
in free or pharmaceutically acceptable salt form.

Further provided is Composition 1 as follows:
1.1 Composition 1, wherein the composition comprises a pharmaceutically acceptable carrier.
1.2 Composition 1 or 1.1, wherein the composition comprises the compound, in free or pharmaceutically acceptable salt form, as described in any of Formula I or 1.1-1.15 vide supra.
1.3 Any of Composition 1, 1.1, or 1.2, wherein the compound is in free form.
1.4 Any of Composition 1 or 1.1-1.3, wherein the compound of Formula I is:

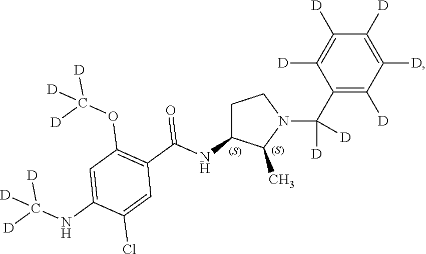

in free or pharmaceutically acceptable salt form, e.g., in free form.
1.5 Any of Composition 1 or 1.1-1.4, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.6 Any of Composition 1 or 1.1-1.5, wherein the compound of Formula I, in free or pharmaceutically acceptable salt form, has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Composition 1 or 1.1-1.5, wherein the compound of Formula I, in free or pharmaceutically acceptable salt form, has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.7 Any of Composition 1 or 1.1-1.6, wherein the composition is in oral or parenteral dosage form, e.g., oral dosage form, for instance, a tablet, capsule, solution, or suspension, for instance, a capsule or tablet.
1.8 Any of Composition 1 or 1.1-1.7, wherein the composition comprises a therapeutically effective amount of the compound of Formula I, in free or pharmaceutically acceptable salt form, e.g., a therapeutically effective amount of the compound of Formula I, in free or pharmaceutically acceptable salt form, for the prophylaxis or treatment of a disorder disclosed herein, e.g., a therapeutically effective amount of the compound of Formula I, in free or pharmaceutically acceptable salt form, for use in any of the methods disclosed herein.
1.9 Any of Composition 1 or 1.1-1.8, wherein the composition is substantially free of any other stereoisomeric form of Formula I. For instance, any of Composition 1 or 1.1-1.8, wherein the composition is substantially free of any other diastereomeric and/or enantiomeric form of Formula I, e.g., wherein the composition is substantially free of any other diastereomeric and enantiomeric form of Formula I.
1.10 Any of Composition 1 or 1.1-1.9, wherein the composition comprises less than 10% w/w (weight/weight) of any other stereoisomeric form of Formula I, e.g., less than 5% w/w of any other stereoisomeric form of Formula I, e.g., less than 4% w/w of any other stereoisomeric form of Formula I, e.g., less than 3% w/w of any other stereoisomeric form of Formula I, e.g., less than 2% w/w of any other stereoisomeric form of Formula I, e.g., less than 1% w/w of any other stereoisomeric form of Formula I.
1.11 Any of Composition 1 or 1.1-1.10, wherein the composition comprises less than 10% w/w of any other diastereomeric form of Formula I, e.g., less than 5% w/w of any other diastereomeric form of Formula I, e.g., less than 4% w/w of any other diastereomeric form of Formula I, e.g., less than 3% w/w of any other diastereomeric form of Formula I, e.g., less than 2% w/w of any other diastereomeric form of Formula I, e.g., less than 1% w/w of any other diastereomeric form of Formula I.
1.12 Any of Composition 1 or 1.1-1.11, wherein the composition comprises less than 10% w/w of any other enantiomeric form of Formula I, e.g., less than 5% w/w of any other enantiomeric form of Formula I, e.g., less than 4% w/w of any other enantiomeric form of Formula I, e.g., less than 3% w/w of any other enantiomeric form of Formula I, e.g., less than 2% w/w of any other enantiomeric form of Formula I, e.g., less than 1% w/w of any other enantiomeric form of Formula I.
1.13 Any of Composition 1 or 1.1-1.12, wherein the compound has the stereochemical configuration as shown in Formula I.
1.14 Any of Composition 1 or 1.1-1.13, wherein the composition comprises 1-60 mg of the compound of Formula I, in free or pharmaceutically acceptable salt form. For instance, any of Composition 1 or 1.1-1.13, wherein the composition comprises 1-10 mg, e.g., 1-9 mg (e.g., 1-8 mg) of the compound of Formula I, in free or pharmaceutically acceptable salt form. For instance, any of Composition 1 or 1.1-1.13, wherein the composition comprises 3 mg or 10 mg of the compound of Formula I, in free or pharmaceutically acceptable salt form. For instance, any of Composition 1 or 1.1-1.13, wherein the composition comprises 1 mg to less than 3 mg (e.g., 2 mg) of the compound of Formula I, in free or pharmaceutically acceptable salt form.
1.15 Any of Composition 1 or 1.1-1.14, wherein the composition is for once, twice, or three times daily dosing. For instance, any of Composition 1 or 1.1-1.14, wherein the composition is for once daily dosing.

Further provided are methods of prophylaxis or treatment of a central nervous system disorder (e.g., a brain disorder), for instance, a central nervous system disorder (e.g., a brain disorder) that benefits from modulating dopamine, in a patient (e.g., a human) in need thereof, wherein the method comprises administering to the patient a compound of Formula X, in free or pharmaceutically acceptable salt form, or a compound of Formula I, in free or pharmaceutically acceptable salt form (e.g., any of Formula I or 1.1-1.15 vide supra), or a pharmaceutical composition comprising a compound of Formula X, in free or pharmaceutically acceptable salt form, or a pharmaceutical composition comprising a compound of Formula I, in free or pharmaceutically acceptable salt form (e.g., Formula 1.15 or any of Composition 1 or 1.1-1.15 vide supra), or Compound A, in free or pharmaceutically acceptable salt form (vide infra), or a pharmaceutical composition comprising Compound A, in free or pharmaceutically acceptable salt form (vide infra). Further provided are methods of prophylaxis or treatment of a central nervous system disorder (e.g., a brain disorder) that benefits from D2 receptor antagonism, D3 receptor antagonism, and/or D4 receptor antagonism in a patient (e.g., a human) in need thereof, wherein the method comprises administering to the patient a compound of Formula X, in free or pharmaceutically acceptable salt form, or a compound of Formula I, in free or pharmaceutically acceptable salt form (e.g., any of Formula I or 1.1-1.15 vide supra), or a pharmaceutical composition comprising a compound of Formula X, in free or pharmaceutically acceptable salt form, or a pharmaceutical composition comprising a compound of Formula I, in free or pharmaceutically acceptable salt form (e.g., Formula 1.15 or any of Composition 1 or 1.1-1.15 vide supra), or Compound A, in free or pharmaceutically acceptable salt form (vide infra), or a pharmaceutical composition comprising Compound A, in free or pharmaceutically acceptable salt form (vide infra). For instance, provided are methods as described below.

Provided are any of the methods below for treatment or prophylaxis of a disorder (e.g., a brain disorder) in a patient (e.g., a human) in need thereof, wherein the method comprises administering to the patient an effective amount of a compound of Formula X, in free or pharmaceutically acceptable salt form.

Provided is a method (Method 1) for treatment or prophylaxis of a disorder (e.g., a brain disorder) in a patient (e.g., a human) in need thereof, wherein the method comprises administering to the patient an effective amount of a compound of Formula I:

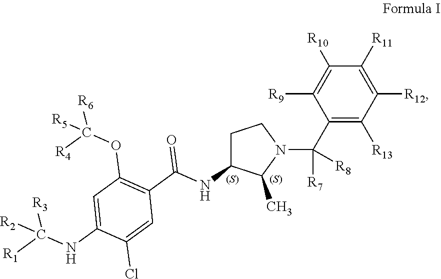

wherein:
R₁, R₂, R₃, R₄, R₅, R₆, R₇, R₈, R₉, R₁₀, R₁₁, R₁₂, and R₁₃ are independently selected from H and D;
and wherein
at least one of R₁, R₂, and R₃, is D;
and at least one of R₉, R₁₀, R₁₁, R₁₂, and R₁₃ is D;
in free or pharmaceutically acceptable salt form.

Further provided is Method 1 as follows:
1.1 Method 1, wherein the method comprises administering to the patient a compound of Formula I, in free or pharmaceutically acceptable salt form, as described in any of Formula I or 1.1-1.15 vide supra. For instance, Method 1, wherein the method comprises administering to the patient a pharmaceutical composition comprising a compound of Formula I, in free or pharmaceutically acceptable salt form, as described in any of Composition 1 or 1.1-1.15 vide supra.
1.2 Method 1 or 1.1, wherein the effective amount of the compound of Formula I, in free or pharmaceutically acceptable salt form, has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the effective amount of the compound of Formula I, in free or pharmaceutically acceptable salt form, is substantially diastereomerically and/or enantiomerically pure, e.g., wherein the effective amount of the compound of Formula I, in free or pharmaceutically acceptable salt form, is substantially diastereomerically and enantiomerically pure. For instance, wherein the effective amount of the compound of Formula I, in free or pharmaceutically acceptable salt form, has a diastereomeric and/or enantiomeric excess of greater than 90%, e.g., a diastereomeric and/or enantiomeric excess equal to or greater than 95%, a diastereomeric and/or enantiomeric excess equal to or greater than 96%, a diastereomeric and/or enantiomeric excess equal to or greater than 97%, e.g., a diastereomeric and/or enantiomeric excess equal to or greater than 98%, e.g., a diastereomeric and/or enantiomeric excess equal to or greater than 99%. For instance, wherein the effective amount of the compound of Formula I, in free or pharmaceutically acceptable salt form, has a diastereomeric and enantiomeric excess of greater than 90%, e.g., a diastereomeric and enantiomeric excess equal to or greater than 95%, e.g., a diastereomeric and enantiomeric excess equal to or greater than 96%, e.g., a diastereomeric and enantiomeric excess equal to or greater than 97%, e.g., a diastereomeric and enantiomeric excess equal to or greater than 98%, e.g., a diastereomeric and enantiomeric excess equal to or greater than 99%.
1.3 Any of Method 1, 1.1, or 1.2, wherein the compound is in free form.
1.4 Any of Method 1 or 1.1-1.3, wherein the method comprises administering an effective amount of Compound A:

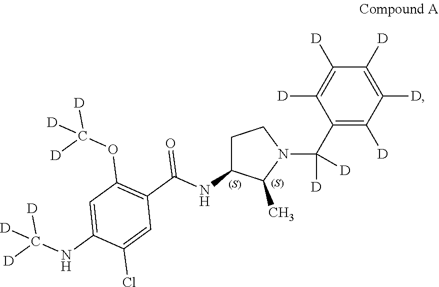

in free or pharmaceutically acceptable salt form, e.g., in free form.
1.5 Method 1.4, wherein the effective amount Compound A, in free or pharmaceutically acceptable salt form, has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the effective amount of Compound A, in free or pharmaceutically acceptable salt form, is substantially diastereomerically and/or enantiomerically pure, e.g., wherein the effective amount of Compound A, in free or pharmaceutically acceptable salt form, is substantially diastereomerically and enantiomerically pure. For instance, wherein the effective amount of Compound A, in free or pharmaceutically acceptable salt form, has a diastereomeric and/or enantiomeric excess of greater than 90%, e.g., a diastereomeric and/or enantiomeric excess equal to or greater than 95%, e.g., a diastereomeric and/or enantiomeric excess equal to or greater than 96%, e.g., a diastereomeric and/or enantiomeric excess equal to or greater than 97%, e.g., a diastereomeric and/or enantiomeric excess equal to or greater than 98%, e.g., a diastereomeric and/or enantiomeric excess equal to or greater than 99%. For instance, wherein the effective amount of Compound A, in free or pharmaceutically acceptable salt form, has a diastereomeric and enantiomeric excess of greater than 90%, e.g., a diastereomeric and enantiomeric excess equal to or greater than 95%, e.g., a diastereomeric and enantiomeric excess equal to or greater than 96%, e.g., a diastereomeric and enantiomeric excess equal to or greater than 97%, e.g., a diastereomeric and enantiomeric excess equal to or greater than 98%, e.g., a diastereomeric and enantiomeric excess equal to or greater than 99%.
1.6 Any of Method 1 or 1.1-1.5, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.7 Any of Method 1 or 1.1-1.6, wherein the effective amount of the compound, in free or pharmaceutically acceptable salt form, has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Method 1 or 1.1-1.6, wherein the effective amount of the compound, in free or pharmaceutically acceptable salt form, has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.8 Any of Method 1 or 1.1-1.7, wherein the disorder is a brain disorder. For instance, any of Method 1 or 1.1-1.7, wherein the disorder is a neuropsychiatric condition in which anhedonia is prominent.
1.9 Any of Method 1 or 1.1-1.8, wherein the disorder is an affective (mood) disorder or an anxiety disorder.
1.10 Any of Method 1 or 1.1-1.9, wherein the disorder is depression (e.g., depression associated with anhedonia), an anxiety disorder, psychosis (e.g., psychosis in neurodegenerative conditions, such as psychosis in Alzheimer's disease, Parkinson's disease, or dementia (e.g., dementia-related psychosis)), schizophrenia, schizoaffective disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder (including bipolar depression, bipolar mania, and bipolar disorder with mixed features), or a migraine. For instance, any of Method 1 or 1.1-1.9, wherein the anxiety disorder is panic disorder, social anxiety disorder, a phobia, or generalized anxiety disorder. Or, any of Method 1 or 1.1-1.9, wherein the method is prophylaxis or treatment of behavioral and psychological symptoms of dementia including agitation, depression, anxiety, apathy, and/or psychosis.
1.11 Any of Method 1 or 1.1-1.10, wherein the disorder is anhedonia or depression associated with anhedonia, suicidal ideation, anxious depression, inflammatory depression, treatment-resistant depression, dysthymia, bipolar depression, psychotic depression, or post-psychotic depression. For instance, any of Method 1 or 1.1-1.10, wherein the disorder is depression associated with anhedonia. Or, for instance, wherein the disorder is anhedonia. Or, for instance, any of Method 1 or 1.1-1.10, wherein the disorder is melancholic depression.
1.12 Any of Method 1 or 1.1-1.11, wherein the disorder is major depressive disorder.
1.13 Any of Method 1 or 1.1-1.10, wherein the disorder is a substance use disorder.
1.14 Any of Method 1 or 1.1-1.10, wherein the method is prophylaxis or treatment of negative symptoms of schizophrenia. Or, any of Method 1 or 1.1-1.10, wherein the method is improving cognition in schizophrenia.
1.15 Any of Method 1 or 1.1-1.10, wherein the method is improving cognition, e.g., in cognitive impairment, e.g., cognitive impairment in schizophrenia or depression. For instance, any of Method 1 or 1.1-1.10, wherein the method is improving cognition in major depressive disorder.
1.16 Any of Method 1 or 1.1-1.7, wherein the compound, in free or pharmaceutically acceptable salt form, is administered as an anti-emetic.
1.17 Any of Method 1 or 1.1-1.16, wherein the method comprises administering 9-60 mg a day of the compound, in free or pharmaceutically acceptable salt form (i.e., 9-60 mg total daily dose of the compound, in free or pharmaceutically acceptable salt form). For instance, any of Method 1 or 1.1-1.16, wherein the method comprises administering 9-36 mg a day of the compound, in free or pharmaceutically acceptable salt form (i.e., 9-36 mg total daily dose of the compound, in free or pharmaceutically acceptable salt form).
1.18 Any of Method 1 or 1.1-1.17, wherein the method comprises administering an amount of the compound, in free or pharmaceutically acceptable salt form, that provides 55%-80% D2/D3 receptor occupancy, e.g., as measured by positron emission tomography. For instance, wherein the method comprises administering an amount of the compound, in free or pharmaceutically acceptable salt form, that provides about 65% D2/D3 receptor occupancy, e.g., as measured by positron emission tomography. Or, for instance, wherein the method comprises administering an amount of the compound, in free or pharmaceutically acceptable salt form, that provides about 60% D2/D3 receptor occupancy, e.g., as measured by positron emission tomography.
1.19 Method 1.17 or 1.18, wherein the disorder is psychosis (e.g., psychosis in neurodegenerative conditions, such as Alzheimer's disease, Parkinson's disease, and dementia (e.g., dementia-related psychosis)), schizophrenia, schizoaffective disorder, or bipolar disorder (e.g., bipolar mania).
1.20 Method 1.17 or 1.18, wherein the method is prophylaxis or treatment of negative symptoms of schizophrenia. Or, Method 1.17 or 1.18, wherein the method is improving cognition in schizophrenia.
1.21 Any of Method 1 or 1.1-1.16, wherein the method comprises administering 1-9 mg (e.g., 1-8 mg, e.g., 1.5-6 mg) a day of the compound, in free or pharmaceutically acceptable salt form (i.e., 1-9 mg total daily dose, e.g., 1-8 mg total daily dose, e.g., 1.5-6 mg total daily dose, of the compound, in free or pharmaceutically acceptable salt form). For instance, any of Method 1 or 1.1-1.16, wherein the method comprises administering 1-8 mg a day of the compound, in free or pharmaceutically acceptable salt form (i.e., 1-8 mg total daily dose of the compound, in free or pharmaceutically acceptable salt form). For instance, any of Method 1 or 1.1-1.16, wherein the method comprises administering 1-3 mg a day of the compound, in free or pharmaceutically acceptable salt form (i.e., 1-3 mg total daily dose of the compound, in free or pharmaceutically acceptable salt form). For instance, any of Method 1 or 1.1-1.16, wherein the method comprises administering 1 mg to less than 3 mg a day (e.g., 2 mg a day) of the compound, in free or pharmaceutically acceptable salt form (i.e., 1 mg to less than 3 mg total daily dose of the compound, in free or pharmaceutically acceptable salt form).
1.22 Any of Method 1, 1.1-1.16, or 1.21, wherein the method comprises administering an amount of the compound, in free or pharmaceutically acceptable salt form, that provides 10%-60% (e.g., 40%-60% or, e.g., 10%-55%, e.g., 10%-50%, e.g., 30%-50% or, e.g., 15%-50%, e.g., 15%-45%, e.g., 20%-40%, e.g., 10%-30%) D2/D3 receptor occupancy, e.g., as measured by positron emission tomography. Or, for instance, any of Method 1, 1.1-1.16, or 1.21, wherein the method comprises administering an amount of the compound, in free or pharmaceutically acceptable salt form, that provides ≤40% (e.g., about 40%), e.g., <40% D2/D3 receptor occupancy, e.g., as measured by positron emission tomography.
1.23 Method 1.21 or 1.22, wherein the disorder is depression (e.g., depression associated with anhedonia), an anxiety disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder, bipolar disorder with mixed features, or a migraine. For instance, Method 1.21 or 1.22, wherein the anxiety disorder is panic disorder, social anxiety disorder, a phobia, or generalized anxiety disorder.
1.24 Any of Method 1.21-1.23, wherein the disorder is anhedonia or depression associated with anhedonia, suicidal ideation, anxious depression, inflammatory depression, treatment-resistant depression, dysthymia, bipolar depression, psychotic depression, or post-psychotic depression. For instance, wherein the disorder is anhedonia or depression associated with anhedonia.
1.25 Any of Method 1.21-1.24, wherein the disorder is major depressive disorder.
1.26 Method 1.21 or 1.22, wherein the disorder is a substance use disorder.
1.27 Any of Method 1 or 1.1-1.26, wherein the method comprises administering a pharmaceutical composition comprising the compound, in free or pharmaceutically acceptable salt form. For instance, any of Method 1 or 1.1-1.26, wherein the method comprises administering Formula 1.15 or any of Composition 1 or 1.1-1.15 vide supra.
1.28 Any of Method 1 or 1.1-1.27, wherein the method comprises administering the compound of Formula I, in free or pharmaceutically acceptable salt form, once, twice, or three times a day, e.g., once a day. For instance, any of Method 1 or 1.1-1.27, wherein the method comprises administering a pharmaceutical composition comprising the compound of Formula I, in free or pharmaceutically acceptable salt form, once, twice, or three times a day, e.g., once a day.
1.29 Any of Method 1 or 1.1-1.28, wherein the method comprises administering Compound A, in free or pharmaceutically acceptable salt form, once, twice, or three times a day, e.g., once a day. For instance, any of Method 1 or 1.1-1.28, wherein the method comprises administering a pharmaceutical composition comprising Compound A, in free or pharmaceutically acceptable salt form, once, twice, or three times a day, e.g., once a day.

Further provided is a compound of Formula X or Formula I (e.g., any of Formula 1.1-1.15) or a pharmaceutical composition disclosed herein (e.g., Formula 1.15 or any of Composition 1 or 1.1-1.15) for use in any of Method 1 or 1.1-1.29 vide supra.

Further provided is use of a compound of Formula X or Formula I (e.g., any of Formula 1.1-1.15) or a pharmaceutical composition disclosed herein (e.g., Formula 1.15 or any of Composition 1 or 1.1-1.15) in any of Method 1 or 1.1-1.29 vide supra.

Further provided is use of a compound of Formula X or Formula I (e.g., any of Formula 1.1-1.15) in the manufacture of a medicament (e.g., Formula 1.15 or any of Composition 1 or 1.1-1.15) for use in any of Method 1 or 1.1-1.29 vide supra.

Further provided are intermediate compounds of Formula II and Formula III, each in free or salt (e.g., pharmaceutically acceptable salt) form.

For instance, further provided is a compound of Formula II:

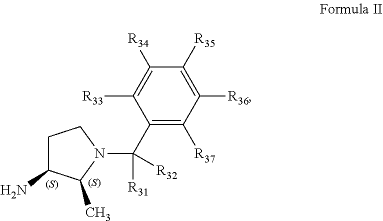

wherein:
R₃₁, R₃₂, R₃₃, R₃₄, R₃₅, R₃₆, and R₃₇ are independently selected from H and D; and
at least one of R₃₃, R₃₄, R₃₅, R₃₆, and R₃₇ is D;
in free or salt form,
optionally wherein the compound is substantially free of its (R,R) enantiomer.

Further provided are compounds of Formula II as follows:
2.1 Formula II, wherein the compound is in pharmaceutically acceptable salt form.
2.2 Formula II or 2.1, wherein R₃₁ and R₃₂ are D.
2.3 Any of Formula II, 2.1, or 2.2, wherein each of R₃₃, R₃₄, R₃₅, R₃₆, and R₃₇ are D.
2.4 Any of Formula II or 2.1-2.3, wherein each of R₃₁, R₃₂, R₃₃, R₃₄, R₃₅, R₃₆, and R₃₇ are D.
2.5 Any of Formula II or 2.1-2.4, wherein the compound is:

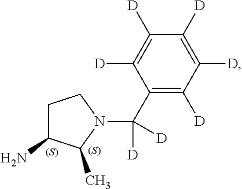

in free or salt (e.g., pharmaceutically acceptable salt) form, e.g., in free form.

Also further provided is a compound of Formula III:

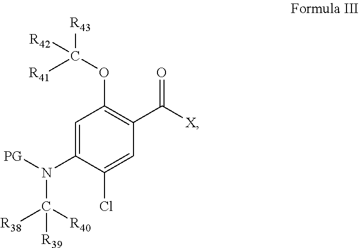

wherein:
X is OH or a leaving group;
PG is H or an amine protecting group (e.g., an ester, which with the nitrogen to which it is attached forms a carbamate, e.g., tert-butyloxycarbonyl or carboxybenzyl, e.g., tert-butyloxycarbonyl);
R₃₈, R₃₉, R₄₀, R₄₁, R₄₂, and R₄₃ are independently selected from H and D; and
at least one of R₃₈, R₃₉, and R₄₀ is D;
in free or salt form.

Further provided are compounds of Formula III as follows:
3.1 Formula III, wherein the compound is in pharmaceutically acceptable salt form.
3.2 Formula III or 3.1, wherein each of R₃₈, R₃₉, and R₄₀ are D.
3.3 Any of Formula III, 3.1, or 3.2, wherein each of R₄₁, R₄₂, and R₄₃ are D.
3.4 Any of Formula III or 3.1-3.3, wherein each of R₃₈, R₃₉, R₄₀, R₄₁, R₄₂, and R₄₃ are D.
3.5 Any of Formula III or 3.1-3.4, wherein the compound is:

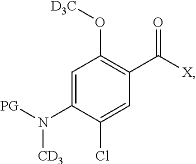

in free or salt (e.g., pharmaceutically acceptable salt) form, e.g., in free form.
3.6 Any of Formula III or 3.1-3.5, wherein the compound is:

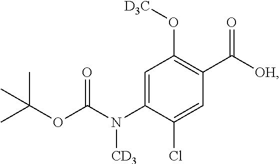 [SMILES: [2H]C([2H])([2H])Oc1cc(N(C(=O)OC(C)(C)C)C([2H])([2H])[2H])c(Cl)cc1C(=O)O]

in free or salt (e.g., pharmaceutically acceptable salt) form, e.g., in free form.
3.7 Any of Formula III or 3.1-3.5, wherein X is OH.
3.8 Any of Formula III or 3.1-3.5, wherein X is a leaving group (e.g., an activated ester, e.g., an O-acylisourea, or a halide). For instance, any of Formula III or 3.1-3.5, wherein the compound is:

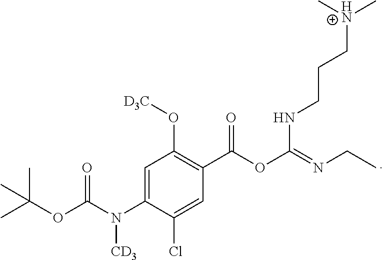

Further provided is a process (Process 1) for synthesizing a compound of Formula I (e.g., any of Formula 1.1-1.15), in free or salt (e.g., pharmaceutically acceptable salt) form.

Further provided is Process 1 as follows:
1.1 Process 1, wherein the process comprises reacting a compound of Formula II (e.g., any of Formula 2.1-2.5) with a compound of Formula III (e.g., any of Formula 3.1-3.8).
1.2 Process 1 or 1.1, wherein the process occurs in the presence of an amine (e.g., triethylamine, dimethylformamide, and/or dimethylacetamide).
1.3 Any of Process 1, 1.1, or 1.2, wherein the process occurs in an organic solvent (e.g., dimethylformamide, triethylamine, and/or dimethylacetamide).
1.4 Any of Process 1 or 1.1-1.3, wherein the process occurs with 1-ethyl-3-(3-dimethylaminopropyl)carbodiimide and hydroxybenzotriazole. For instance, any process wherein the process occurs with 1-ethyl-3-(3-dimethylaminopropyl)carbodiimide, hydroxybenzotriazole, triethylamine, and dimethylformamide and/or dimethylacetamide.
1.5 Any of Process 1 or 1.1-1.4, wherein the process comprises reacting a compound of Formula IIIa:

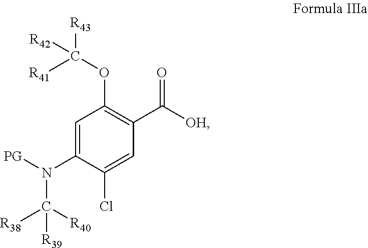

wherein PG is H or an amine protecting group (e.g., H); and
R₃₈, R₃₉, R₄₀, R₄₁, R₄₂, and R₄₃ are independently selected from H and D and at least one of R₃₈, R₃₉, and R₄₀ is D, in free or salt (e.g., pharmaceutically acceptable salt) form, with an activating agent (e.g., 1-ethyl-3-(3-dimethylaminopropyl)carbodiimide).
1.6 Process 1.5, wherein the process forms a compound of Formula IIIb:

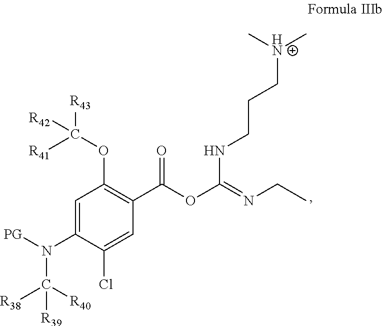

wherein PG is H or an amine protecting group (e.g., H); and
R₃₈, R₃₉, R₄₀, R₄₁, R₄₂, and R₄₃ are independently selected from H and D and at least one of R₃₈, R₃₉, and R₄₀ is D, in free or salt (e.g., pharmaceutically acceptable salt) form.
1.7 Process 1.6, wherein the compound of Formula IIIb is formed in situ.
1.8 Any of Process 1 or 1.1-1.7, further comprising removing the amine protecting group, e.g., under acidic or basic conditions, e.g., with HCl optionally in ethyl acetate.
1.9 Any of Process 1 or 1.1-1.8, wherein the process further comprises isolating the compound of Formula I (e.g., any of Formula 1.1-1.15), in free or salt (e.g., pharmaceutically acceptable salt) form.

For compounds disclosed herein, a hydrogen atom position of a structure is considered substituted with deuterium when the abundance of deuterium at that position is enriched. The natural abundance of deuterium is about 0.02%, so a compound is “enriched” with deuterium at a specific position when the frequency of incorporation of deuterium at that position exceeds 0.02%. Therefore, for deuterated compounds disclosed herein, any position designated as deuterium (i.e., D) may be enriched with deuterium at a level of greater than 0.1%, or greater than 0.5%, or greater than 1%, or greater than 5%, such as, greater than 50%, or greater than 60%, or greater than 70%, or greater than 80%, or greater than 90%, or greater than 95%, or greater than 96%, or greater than 97%, or greater than 98%, or greater than 99%. For compounds disclosed herein, any atom not designated as a particular isotope is present at natural isotopic abundance.

Compounds disclosed herein, e.g., any of Formula X, Formula I (e.g., any of Formula 1.1-1.15), Formula II (e.g., any of Formula 2.1-2.5), Formula III (e.g., any of 3.1-3.8), and Compound A, may exist in free or salt form, e.g., as acid addition salts. As used herein, unless otherwise indicated, language such as “compound of formula” is to be understood as embracing the compound in any form, for example free or acid addition salt form, or where the compound contains an acidic substituent, in base addition salt form. Compounds of Formula X, Formula I (e.g., any of Formula 1.1-1.15), and Compound A are intended for use as pharmaceuticals, therefore pharmaceutically acceptable salts are preferred. Salts which are unsuitable for pharmaceutical uses may be useful, for example, for the isolation or purification of free compounds of Formula X, Formula I (e.g., any of Formula 1.1-1.15), or Compound A or their pharmaceutically acceptable salts, so therefore are also included.

Isolation or purification of the stereoisomers of compounds disclosed herein, for instance, Formula X, Formula I (e.g., any of Formula 1.1-1.15), Formula II (e.g., any of Formula 2.1-2.5), Formula III (e.g., any of 3.1-3.8), and Compound A, any in free or pharmaceutically acceptable salt form, may be achieved by conventional methods known in the art, e.g., column purification, preparative thin layer chromatography, preparative HPLC, trituration, simulated moving beds, and the like.

Pure stereoisomeric forms of the compounds and intermediates disclosed herein are isomers substantially free of other enantiomeric and diastereomeric forms of the same basic molecular structure of said compounds or intermediates. “Substantially stereoisomerically pure” includes compounds or intermediates having a stereoisomeric excess of greater than 90% (i.e., more than 90% of one stereoisomer and less than 10% of any other possible stereoisomer). The terms “substantially diastereomerically pure” and “substantially enantiomerically pure” should be understood in a similar way, but then having regard to the diastereomeric excess and enantiomeric excess, respectively, of the material in question.

Compounds disclosed herein, e.g., any of Formula X, Formula I (e.g., any of Formula 1.1-1.15), Formula II (e.g., any of Formula 2.1-2.5), Formula III (e.g., any of 3.1-3.8), and Compound A, any in free or pharmaceutically acceptable salt form, may be made by using the methods as described and exemplified herein and by methods similar thereto and by methods known in the chemical art. Such methods include, but are not limited to, those described below. If not commercially available, starting materials for these processes may be made by procedures, which are selected from the chemical art using techniques that are similar to or analogous to the synthesis of known compounds.

Pharmaceutically acceptable salts of any of Formula X, Formula I (e.g., any of Formula 1.1-1.15), Formula II (e.g., any of Formula 2.1-2.5), Formula III (e.g., any of 3.1-3.8), and Compound A, may be synthesized from the parent compound which contains a basic or acidic moiety by conventional chemical methods. Generally, such salts can be prepared by reacting the free base forms of these compounds with a stoichiometric amount of the appropriate acid in an appropriate solvent.

For methods of treatment, the word “effective amount” is intended to encompass a therapeutically effective amount to treat a specific disease or disorder.

Dosages employed in practicing the present invention will of course vary depending, e.g. on the particular disease or condition to be treated, the particular compound used, the mode of administration, and the therapy desired.

Compounds disclosed herein, e.g., any of Formula X, Formula I (e.g., any of Formula 1.1-1.15), and Compound A, any in free or pharmaceutically acceptable salt form, may be administered by any suitable route, including orally, parenterally, or transdermally, but are preferably administered orally.

Pharmaceutical compositions comprising compounds disclosed herein, e.g., any of Formula X, Formula I (e.g., any of Formula 1.1-1.15 or any of Composition 1 or 1.1-1.15), or Compound A, any in free or pharmaceutically acceptable salt form, may be prepared using conventional diluents or excipients and techniques known in the galenic art. Thus oral dosage forms may include tablets, capsules, solutions, suspensions, and the like.

“Patient” as used herein includes human and non-human (i.e., animal). In some embodiments, the patient is human.

### EXAMPLES

### Abbreviations

AcOH=acetic acid
Boc=tert-butyloxycarbonyl
DIAD=diisopropyl azodicarboxylate
DCM=dichloromethane
DMA or DMAc=dimethylacetamide
DMAP=4-dimethylaminopyridine
DMF=dimethylformamide
EDCI=1-ethyl-3-(3-dimethylaminopropyl)carbodiimide
EtOAc or EA=ethyl acetate
h=hour(s)
HOBt=hydroxybenzotriazole
MeOH=methanol
MsC1=methanesulfonyl chloride
rt (or RT or r.t.)=room temperature
TEA=triethylamine
TFA=trifluoroacetic acid
THF=tetrahydrofuran Example 1

### Synthesis: 5-Chloro-N-((2S,3S)-1-(dideutero(pentadeuterophenyl)methyl)-2-methylpyrrolidin-3-yl)-2-trideuteromethoxy-4-(trideuteromethylamino)benzamide

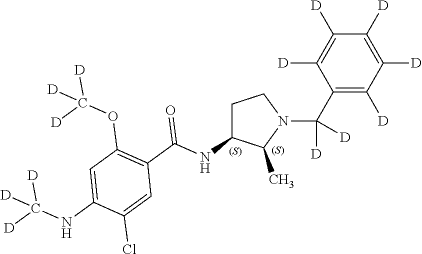

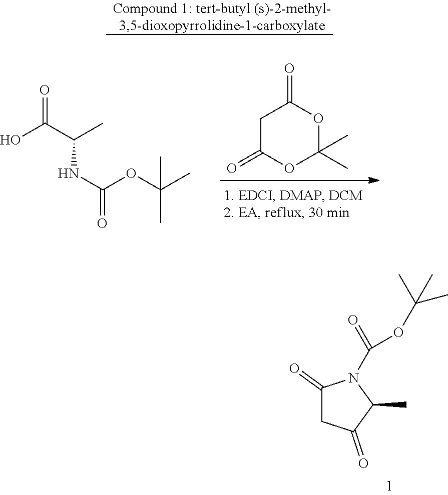

To a stirred solution of Boc-L-alanine (25 g, 132.1 mmol), Meldrum's acid (20 g, 138.7 mmol), and DMAP (19.4 g, 158.6 mmol) in CH₂Cl₂ (250 mL) is added EDCI (30.4 g, 158.6 mmol) under nitrogen at 0° C. The resulting solution is then allowed to warm up to room temperature (rt) and stirred over 16 h. It is quenched with water (50 mL), the organic phase is washed with a cold solution of 5% KHSO₄ (300 mL×2), water (300 mL×1) and brine, then dried over anhydrous MgSO₄, and concentrated to give the residue (40 g). EtOAc (200 mL) is added and the reaction mixture is refluxed for 30 minutes. The solution is concentrated and the residue is stirred in EtOAc (90 mL) at −10° C. for 2 h, then filtered, and the filter cake is collected to give the title compound as a pale yellow solid (16 g, 46% yield). ¹H NMR (400 MHz, CDCl₃) δ 4.41 (q, J=6.8 Hz, 1H), 3.22 (s, 2H), 1.57 (s, 9H), 1.51 (d, J=6.8 Hz, 3H). MS m/z (ESI): 158 [M+H−56]⁺

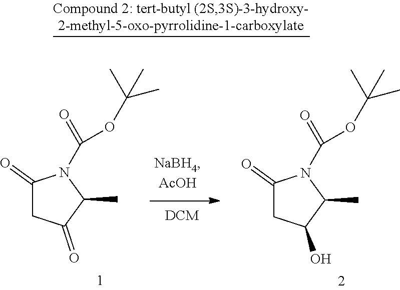

To a stirred solution of compound 1 (13 g, 61 mmol) in dichloromethane (DCM) (130 ml) is added AcOH (65 mL) at 0° C. Then NaBH₄ (5.77 g, 152.4 mmol) is added in three portions. The resulting solution is then allowed to warm up to room temperature and stirred over 16 h. The reaction mixture is quenched with 5% NaHCO₃ at 0° C. It is then extracted with DCM (200 mL×3). The combined organic layers are washed with 5% NaHCO₃, brine, dried over anhydrous MgSO₄, and concentrated to give the residue which is stirred in isopropyl ether to afford the title compound 2 (8 g, 61% yield). ¹H NMR (400 MHz, CDCl₃) δ 4.53-4.47 (m, 1H), 4.29-4.22 (m, 1H), 2.75-2.55 (m, 2H), 1.53 (s, 9H), 1.31 (d, J=6.8 Hz, 3H). MS m/z (ESI): 160 [M+H−56]⁺

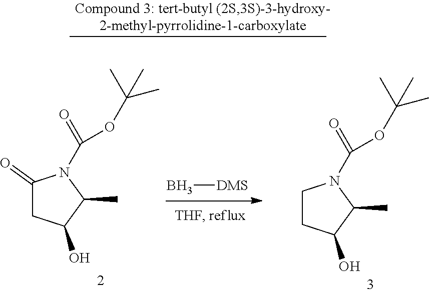

To a solution of compound 2 (3 g, 14 mmol) in dry THE (40 mL) is added a solution of BH₃—SMe₂ (21 ml, 41.8 mmol) at 0° C. and it is stirred for 30 minutes at 0° C. Then the mixture is refluxed for 4 h. The resulting mixture is cooled and quenched with saturated NH₄Cl at 0° C. It is then extracted with EtOAc (100 ml×3). The organic phases are dried over anhydrous MgSO₄ and concentrated to give compound 3 (2.24 g, 80% yield). ¹H NMR (400 MHz, CDCl₃) δ 4.34-4.29 (m, 1H), 3.90-3.83 (m, 1H), 3.46-3.33 (m, 2H), 2.09-1.80 (m, 2H), 1.46 (s, 9H), 1.18 (d, J=6.8 Hz, 3H). MS m/z (ESI): 146 [M+H−56]⁺

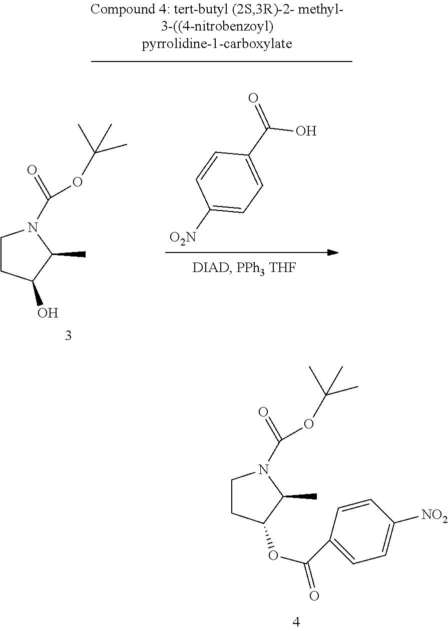 [SMILES: C[C@H]1[C@@H](O)CCN1C(=O)OC(C)(C)C ; O=C(O)c1ccc([N+](=O)[O-])cc1 ; C[C@H]1[C@H](OC(=O)c2ccc([N+](=O)[O-])cc2)CCN1C(=O)OC(C)(C)C]

To a cold solution of compound 3 (12 g, 59.6 mmol), 4-nitrobenzoic acid (10.46 g, 62.6 mmol), and PPh₃ (16.42 g, 62.6 mmol) in dry THE (200 ml) is added diisopropyl azodicarboxylate (DIAD) (12.66 g, 62.6 mmol) for 30 minutes at 0° C. The reaction mixture is allowed to warm room temperature for 16 h. The resulting mixture is cooled and quenched with water. The mixture is extracted with EtOAc (200 ml×3), dried over anhydrous MgSO₄. It is then concentrated and purified by silica gel chromatography to afford the title compound 4 (17.1 g, 81.9% yield). ¹H NMR (400 MHz, CDCl₃) δ 8.31-8.17 (m, 4H), 5.20 (d, J=4 Hz 1H), 4.17-3.86 (m, 1H), 3.59-3.46 (m, 2H), 2.35-2.11 (m, 2H), 1.48 (s, 9H), 1.28 (d, J=6.8 Hz, 3H). MS m/z (ESI): 295 [M+H−56]⁺

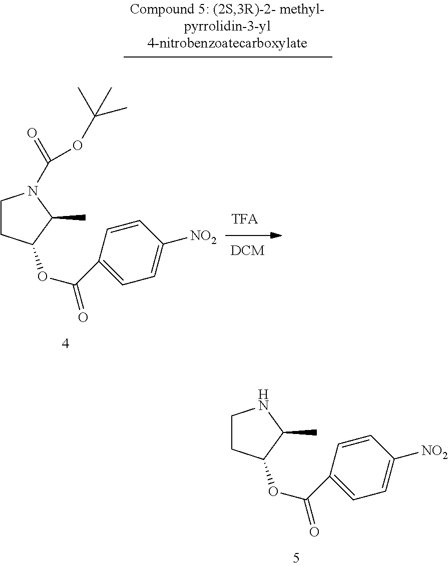 [SMILES: C[C@H]1[C@H](OC(=O)c2ccc([N+](=O)[O-])cc2)CCN1C(=O)OC(C)(C)C ; C[C@@H]1NCC[C@H]1OC(=O)c1ccc([N+](=O)[O-])cc1]

A mixture of compound 4 (16.1 g, 46 mmol) and trifluoroacetic acid (TFA) (80 mL) in DCM (160 mL) is stirred at room temperature for 1 h. The reaction mixture is then concentrated to give the product 5 (11.5 g, 100% yield). MS m/z (ESI): 251 [M+H]⁺

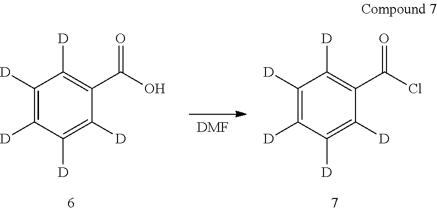

6 (7.5 g, 59 mmol) is dissolved in cold DCM (200 mL, 0° C.). To the solution is added 3 drops of DMF and oxalyl chloride (118 mmol, dropwise). The reaction is allowed to warm to room temperature and is stirred for 3 hours. The solvent is removed under reduced pressure to yield 7 (100% yield, 8.59 g).

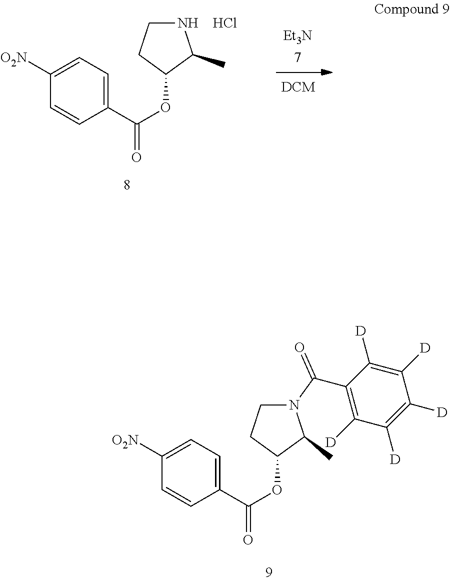

To a solution of 8 (6.0 g, 20.9 mmol) and 7 (4.2 g, 28.9 mmol) in DCM (120 mL) is added Et₃N (9.53 g, 94.2 mmol) at 0° C. The reaction mixture is allowed to warm r.t. and stirred for 16 h. On completion, the reaction mixture us washed with water (70 mL×2), and concentrated to give 9 (7.0 g crude) as a white solid.

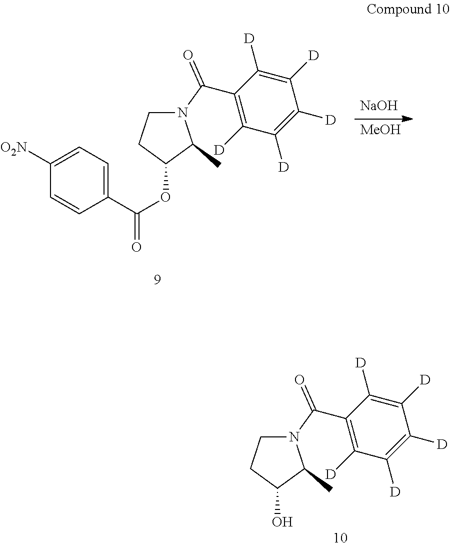

To a stirred solution of 9 (7.7 g, 21.4 mmol) in MeOH/H₂O (39 mL/39 mL) is added NaOH (1.03 g, 25.7 mmol). The reaction mixture is stirred for 2 h and then concentrated under reduced pressure. The residue is diluted with water (32 mL), extracted with DCM (55 mL×5). The organic phase is concentrated to give 10 (3.96 g crude) as a white solid.

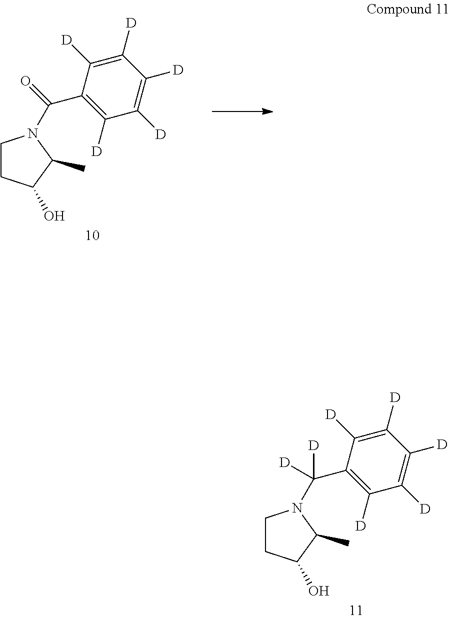 [SMILES: [2H]c1c([2H])c([2H])c(C(=O)N2CC[C@@H](O)[C@@H]2C)c([2H])c1[2H] ; [2H]c1c([2H])c([2H])c(C([2H])([2H])N2CC[C@@H](O)[C@@H]2C)c([2H])c1[2H]]

A solution of 10 in dry THE is added dropwise to a stirred solution of 2.5 equivalents of LiAlD₄ in dry THE (40 mL) at 0-10° C. under nitrogen atmosphere. After stirring at 0˜10° C. for 45 min, the reaction is allowed to warm to room temperature, and stirred at the same temperature over 16 h. Upon completion, it is cooled to 0° C., and quenched with 20% aqueous KOH and H₂O. The suspension is extracted with DCM (2 times). The organic phases are dried over anhydrous Na₂SO₄, filtered and concentrated to give 11.

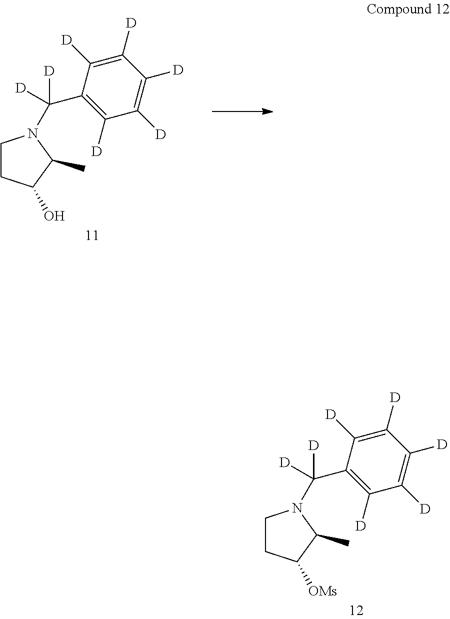 [SMILES: [2H]c1c([2H])c([2H])c(C([2H])([2H])N2CC[C@@H](O)[C@@H]2C)c([2H])c1[2H] ; [2H]c1c([2H])c([2H])c(C([2H])([2H])N2CC[C@@H](OS(C)(=O)=O)[C@@H]2C)c([2H])c1[2H]]

To a stirred solution of 11 and 2 equivalents of Et₃N in DCM at 0° C. is added 1.5 equivalents of MsCl (methanesulfonyl chloride). The reaction mixture is stirred at r.t for 3 h, then quenched with saturated aqueous NaHCO₃ (2 times) and the aqueous layer extracted with DCM. The combined organic phases are washed with brine. The organic phase is concentrated under reduced pressure to give 12.

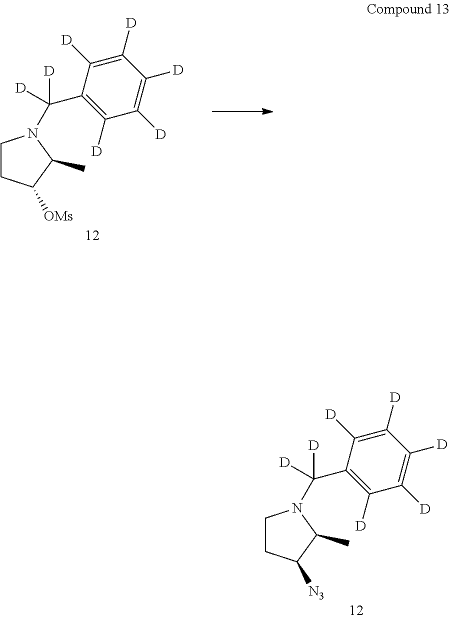 [SMILES: [2H]c1c([2H])c([2H])c(C([2H])([2H])N2CC[C@@H](OS(C)(=O)=O)[C@@H]2C)c([2H])c1[2H] ; [2H]c1c([2H])c([2H])c(C([2H])([2H])N2CC[C@H](N=[N+]=[N-])[C@@H]2C)c([2H])c1[2H]]

To a stirred solution of 12 in DMF is added 3 equivalents of NaN₃ at r.t. The reaction mixture is stirred for 16 h at 80° C. The reaction mixture is quenched with water, extracted with EtOAc (2 times). The organic phase is washed with brine. The organic phase is concentrated to about 1 mL, then MeOH is added and concentrated. The solution of 13 in MeOH is used directly for next step.

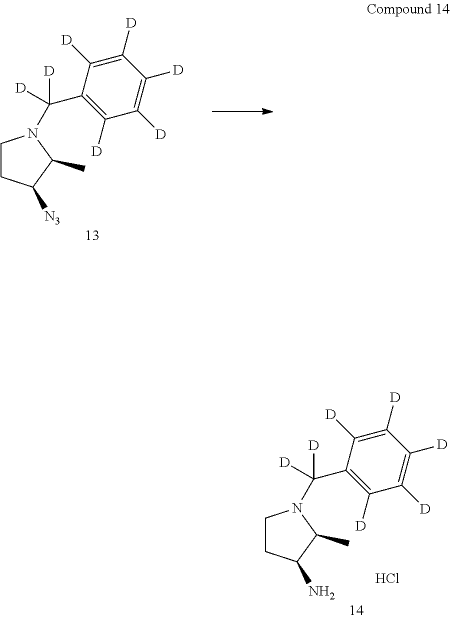 [SMILES: [2H]c1c([2H])c([2H])c(C([2H])([2H])N2CC[C@H](N=[N+]=[N-])[C@@H]2C)c([2H])c1[2H] ; [2H]c1c([2H])c([2H])c(C([2H])([2H])N2CC[C@H](N)[C@@H]2C)c([2H])c1[2H] ; Cl]

A mixture of 13 and 10% of Pd/C in MeOH is stirred under H₂ (atmospheric pressure) over 24 h at r.t. The reaction mixture is filtered and the solvent evaporated, and it is diluted with EtOAc. HCl (4 mol/L in EtOAc) is added to the solution. The reaction mixture is stirred at r.t for 1 h, then filtered to give 14.

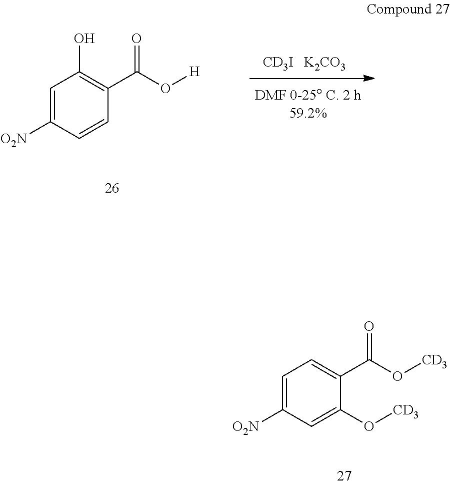 [SMILES: O=C(O)c1ccc([N+](=O)[O-])cc1O ; "CD3I   K2CO3" ; [2H]C([2H])([2H])OC(=O)c1ccc([N+](=O)[O-])cc1OC([2H])([2H])[2H]]

A solution of 26 (9.00 g, 49.2 mmol), CD₃I (17.81 g, 122.9 mmol) and K₂CO₃ (16.98 g, 122.9 mmol) in DMF (90 mL) is stirred at room temperature for 16 hours. The mixture is diluted with water (100 mL) and extracted with EtOAc (100 mL×3). The combined organic layer are washed with brine (100 mL), dried over Na₂SO₄ and concentrated under vacuum to afford 27 (6.32 g, 59.20%) as a brown solid. ¹H NMR (400 MHz, DMSO-d6) δ7.88 (d, J=1.6 Hz, 1H), 7.87-7.85 (m, 2H)

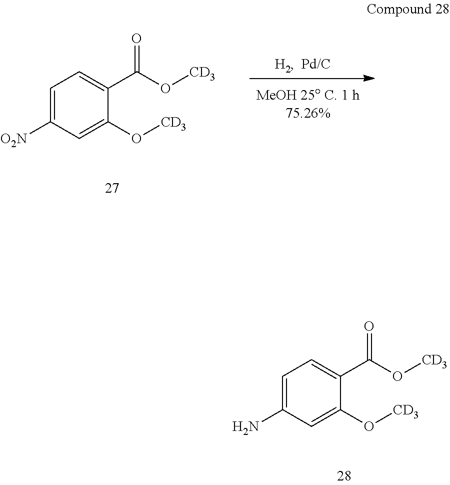 [SMILES: [2H]C([2H])([2H])OC(=O)c1ccc([N+](=O)[O-])cc1OC([2H])([2H])[2H] ; "H2,  Pd/C" ; [2H]C([2H])([2H])OC(=O)c1ccc(N)cc1OC([2H])([2H])[2H]]

To a solution of 27 (6.32 g, 29.1 mmol) in methanol (65 mL) is added 10% Pd—C (100 mg). The reaction mixture is stirred at room temperature for 1 hour under hydrogen (50 psi). Completion of the reaction is monitored by TLC. The mixture is filtered and concentrated under vacuum to afford 28 (4.10 g, 75.26%) as a white solid. LC-MS (ESI) m/z calcd (M+H)⁺187.9, found 187.1

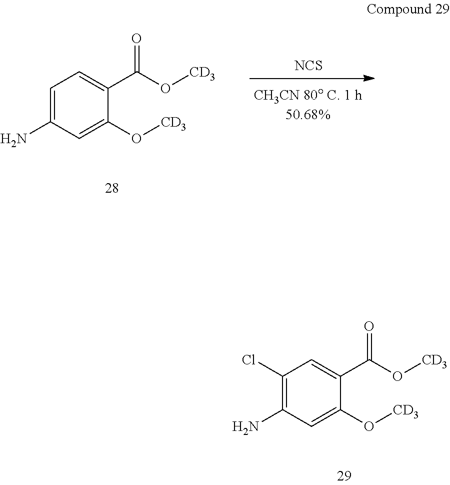 [SMILES: [2H]C([2H])([2H])OC(=O)c1ccc(N)cc1OC([2H])([2H])[2H] ; "NCS" ; [2H]C([2H])([2H])OC(=O)c1cc(Cl)c(N)cc1OC([2H])([2H])[2H]]

To a solution of 28 (4.10 g, 21.9 mmol) in CH₃CN (41 mL) is added NCS (0.85 g, 14.9 mmol). The mixture is stirred at 80° C. for 1 hour. It is cooled to r.t. and concentrated under vacuum. The resulting residue is purified with 1:1 hexanes/EtOAc to give 29 (2.46 g, 50.68%) as a brown solid. LC-MS (ESI) m/z (M+H)+221.9, found 221.1

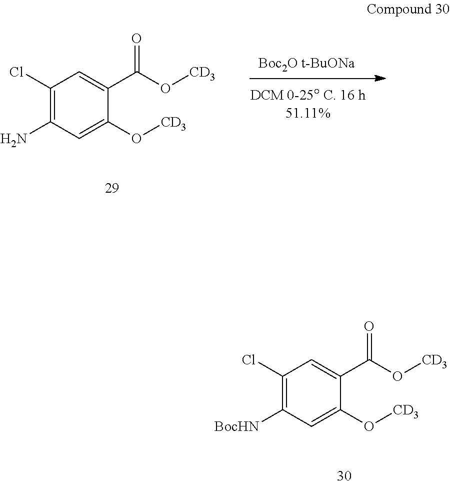 [SMILES: [2H]C([2H])([2H])OC(=O)c1cc(Cl)c(N)cc1OC([2H])([2H])[2H] ; "Boc2O t-BuONa" ; [2H]C([2H])([2H])OC(=O)c1cc(Cl)c(NC(=O)OC(C)(C)C)cc1OC([2H])([2H])[2H]]

To a solution of 29 (2.46 g, 11.1 mmol) in DCM (80 mL) is added t-BuONa (1.60 g, 16.7 mmol) at 0° C. Then Boc₂O (2.42 g, 11.1 mmol) is added dropwise to the mixture. The reaction is stirred at 25° C. for 16 hours. The mixture is concentrated under vacuum to give the residue which is in purified by silica gel chromatography (10-50% EtOAc in petroleum ether) to give 30 (1.83 g) as a yellow solid.

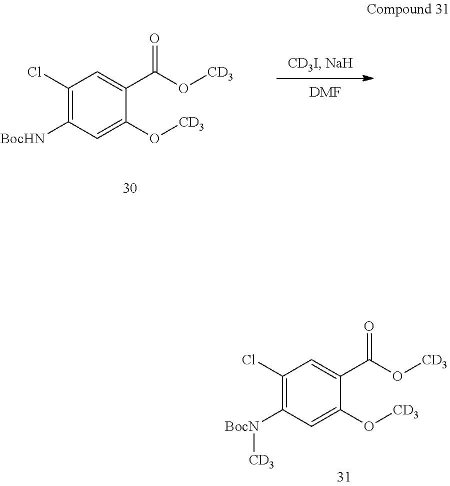

To a solution of 30 in dry DMF is added 1 equivalent of NaH and the resulting solution is stirred at room temperature for 30 minutes. Then 1.5 equivalents of CD₃I is added and the mixture is stirred at room temperature for 3 hours. The reaction is cooled to 0° C. and quenched with saturated aqueous NH₄Cl and extracted with EtOAc. The organic phase is washed with water, brine and dried over anhydrous Na₂SO₄ and concentrated under vacuum. The resulting residue is purified with flash column chromatography to give 31.

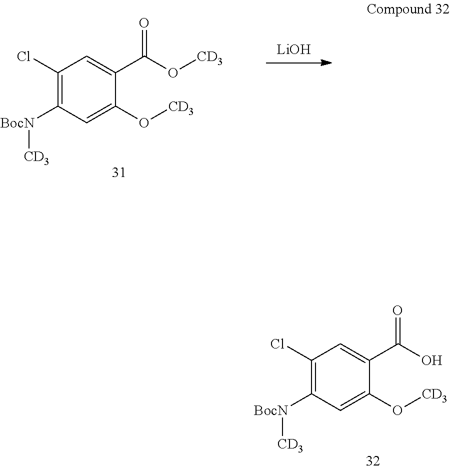 [SMILES: [2H]C([2H])([2H])OC(=O)c1cc(Cl)c(N(C(=O)OC(C)(C)C)C([2H])([2H])[2H])cc1OC([2H])([2H])[2H] ; "LiOH" ; [2H]C([2H])([2H])Oc1cc(N(C(=O)OC(C)(C)C)C([2H])([2H])[2H])c(Cl)cc1C(=O)O]

To a solution of 31 in THF:water (2:1) is added 1.2 equivalents of LiOH and the mixture is stirred at room temperature for 16 hours. Reaction completion is monitored by TLC. The mixture is concentrated under vacuum. The resulting residue is purified by silica gel chromatography (1-12% MeOH in DCM) to give 32.

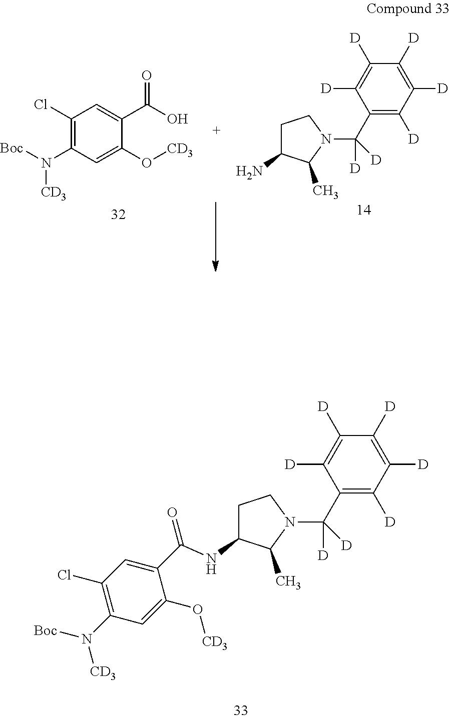 [SMILES: [2H]C([2H])([2H])Oc1cc(N(C(=O)OC(C)(C)C)C([2H])([2H])[2H])c(Cl)cc1C(=O)O ; [2H]c1c([2H])c([2H])c(C([2H])([2H])N2CC[C@H](N)[C@@H]2C)c([2H])c1[2H] ; [2H]c1c([2H])c([2H])c(C([2H])([2H])N2CC[C@H](NC(=O)c3cc(Cl)c(N(C(=O)OC(C)(C)C)C([2H])([2H])[2H])cc3OC([2H])([2H])[2H])[C@@H]2C)c([2H])c1[2H]]

To a stirred solution of 32 in 2 equivalents of Et₃N and DMA is added 1 equivalent of 14, 1.5 equivalents of HOBt, and 1.6 equivalents of EDCI. The reaction mixture is stirred at r.t. for 2 h. The resulting mixture is quenched with water and extracted with EtOAc (3 times), washed with brine (one time) and dried over anhydrous Na₂SO₄. The organic phase is concentrated and the residue is purified by column chromatography (silica gel; 0-10% methanol in dichloromethane) and concentrated to give 33.

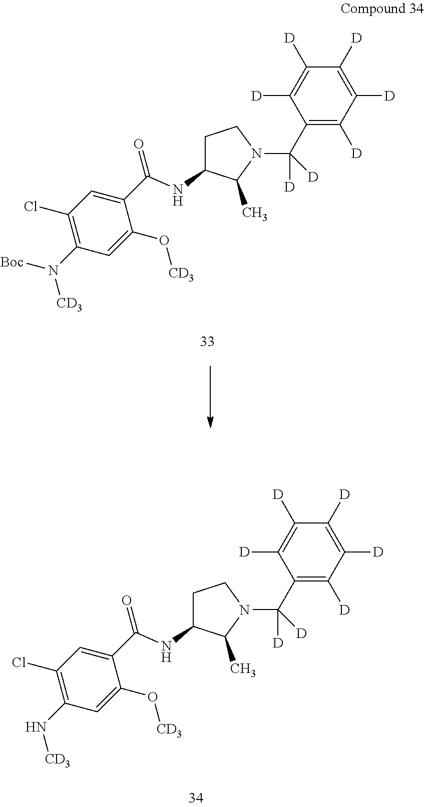 [SMILES: [2H]c1c([2H])c([2H])c(C([2H])([2H])N2CC[C@H](NC(=O)c3cc(Cl)c(N(C(=O)OC(C)(C)C)C([2H])([2H])[2H])cc3OC([2H])([2H])[2H])[C@@H]2C)c([2H])c1[2H] ; [2H]c1c([2H])c([2H])c(C([2H])([2H])N2CC[C@H](NC(=O)c3cc(Cl)c(NC([2H])([2H])[2H])cc3OC([2H])([2H])[2H])[C@@H]2C)c([2H])c1[2H]]

A solution of 33 is stirred in excess HCl (4 mol/L in EtOAc) at r.t. for 2 h. The reaction mixture is concentrated and the residue is diluted with EtOAc, extracted with H₂O (2 times). The aqueous phase is combined and alkalized to pH˜11 with sodium hydroxide, extracted with EtOAc (3 times) and the combined organic phase is washed with brine and dried over anhydrous Na₂SO₄. The organic phase is concentrated and the residue is purified by column chromatography (silica gel, 0-100% EtOAc in hexanes) to afford 34.

### Example 2—Radioligand Binding Competition Activity on Recombinant Human Dopamine and Serotonin Receptors Using Filtration Binding Assays

Radioligand binding experiments are conducted with membrane preparations. Receptor accession numbers, cellular background, and reference compounds are listed in Table 1.

TABLE 1

| Receptor | Accession Number | Cell Line | Reference Tracer | Reference Competitor |
|---|---|---|---|---|
| D2S | NP_057658.2 | CHO-K1 | [³H]-Spiperone | Risperidone |
| D3 | AAA73929.1 | CHO-K1 | [³H]-R-(+)-7-OH-DPAT | R-(+)-7-OH-DPAT |
| D4.4 | NP_000788.2 | CHO-K1 | [³H]-Spiperone | Haloperidol |
| 5-HT1A | NP_000515.2 | CHO-K1 | [³H]-8-OH-DPAT | 5-HT |
| 5-HT2A | NP_000612.1 | CHO-K1 | [³H]-Ketanserin | Ketanserin |
| 5-HT7A | NP_000863.1 | CHO-K1 | [³H]-LSD | 5-CT |
| human D2L | AAB26819.1 | CHO-K1 | [³H]-Spiperone | Risperidone |

The compound from Example 1 (34) is tested for radioligand binding competition activity.

### Example 3—Agonist or Antagonist Activity on Recombinant Human Dopamine and Serotonin Receptors Using IPOne HTRF, cAMP HTRF, and GTPyS Assays

SPA ³⁵S-GTPgS experiments are conducted with membrane preparations. IP-One and cAMP HTRF assays are conducted with recombinant cell lines. Receptor accession numbers, cellular background, and reference compounds are listed in Table 3.

TABLE 3

| Receptor | Accession Number | Assay | Cell Line | Reference Agonist | Reference Antagonist |
|---|---|---|---|---|---|
| D2S | NP_057658.2 | cAMP | CHO-K1 | Quinpirole | Haloperidol |
| D3 | AAA73929.1 | GTP | CHO-K1 | Dopamine | GR103691 |
| D4.4 | NP_000788.2 | cAMP | CHO-K1 | Dopamine | Spiperone |
| 5-HT1A | NP_000515.2 | cAMP | CHO-K1 | 5-CT | Not tested |
| 5-HT2A | NP_000612.1 | IPOne | CHO-K1 | α-Me-5-HT | Ketanserin |
| 5-HT7A | NP_000863.1 | cAMP | CHO-K1 | 5-CT | Risperidone |
| D2L | AAB26819.1 | cAMP | CHO-K1 | Quinpirole | Haloperidol |

The compound from Example 1 (34) is tested for antagonist and agonist activity at human dopamine and serotonin receptors.

### Example 4—In Vivo Pharmacokinetics

Group A rats are dosed (by PO) with test compound. Blood samples are obtained at 5, 10, and 30 minutes, and 1, 2, 4, 8, and 24 hours after dosing. Following blood collection at 24 hours, brain perfusion is performed on the animals before harvesting brain tissues.

Group B rats are dosed (by PO) with test compound. At designated timepoints (1, 4, and 8 hours), three animals from each dose group undergo blood draw followed by brain perfusion before samples are collected.

Test compound is the deuterated compound of Example 1 (34).

Plasma (harvested from blood samples) and brain tissues (homogenized and processed) are analyzed by LC/MS/MS. Plasma is harvested from blood via centrifugation. Brain tissue is collected after animals undergo perfusion to remove residual cardiovascular blood.

### Example 5—Ex Vivo Radioligand Binding in Membrane Preparations to Determine Time-Course of Receptor Occupancy at Central D₂ Receptors

This study is to determine receptor occupancy at central D2 receptors following oral administration of the deuterated compound of Example 1 (34) at various time points (e.g., 1, 2, 4, 8, and 24 hours) and the positive comparator, olanzapine (10 mg/kg, po) using [³H]raclopride and rat striatal membranes. Liquid scintillation counting is used to quantify radioactivity.

### Animals

Rats.

### Drug Treatment

On day of test, animals are dosed orally with either vehicle, a single dose of the deuterated compound of Example 1 (34) or olanzapine. Rats are sacrificed at specified time points, e.g., 1, 2, 4, 8, and 24 hours after drug administration or 1 hour after vehicle and olanzapine administration.

### Pharmacokinetics

A post-mortem blood sample is taken by cardiac puncture. Plasma is taken for PK determination.

Whole brains are removed, rinsed with saline, and blot dried. The left striatum and right striatum is dissected out and weighed before being frozen on dry ice.

### Homogenate Preparation

The striata is homogenised individually.

### Assay

Striatal homogenates are incubated with [³H]raclopride. Radioactivity is determined by liquid scintillation counting.

### Example 6—Touchscreen-Based Rat Probabilistic Reward Task

The Probabilistic Reward Task (PRT) uses visual discrimination methodology to quantify reward responsiveness to both identify deficits and characterize drug-induced improvements. Groups of rats are trained on the touchscreen-based PRT and exposed to asymmetrical probabilistic contingencies to generate response biases to the richly rewarded stimulus (Pizzagalli, D. et al., Biological Psychiatry, 2005, 57, 319-327; Kangas, B. et al., Translational Psychiatry, 2020, 10(1):285; Wooldridge, L. et al., International Journal of Neuropsychopharmacology, 2021, 24, 409-418). Next, subjects are tested with vehicle or the deuterated compound of Example 1 (34).

Details and schematics of the rodent touch-sensitive experimental chamber and methods can be found in Kangas, B. et al., Behavioural Pharmacology, 2017, 28, 623-629.

### Example 7—Conditioned Avoidance Response

Rats are used. Risperidone (0.5 mg/kg; Sigma Aldrich) is dissolved in 10% DMSO in water and injected i.p. at a dose volume of 1 mg/kg 30 minutes prior to test. The deuterated compound of Example 1 (34) is administered orally prior to test.

The Conditioned Avoidance Response (CAR) Test is an animal model screening for antipsychotic drugs.

### Example 8—Headshake Response

Rats are used. The deuterated compound of Example 1 (34) is administered orally.

Animals are administered vehicle, DOI, or test compound and returned to their holding cage for the appropriate pretreatment time, following which headshakes are recorded. The headshake response is a rapid, rhythmic shaking of the head in a radial motion.

### Example 9—DOI-Induced Headshake Response

Rats are used. The deuterated compound of Example 1 (34) is administered orally. DOI is administered IP. Ketanserin (1 mg/kg) is injected IP.

Animals are administered vehicle, ketanserin, or test compound and returned to their holding cage for the appropriate pretreatment time. Rats are then injected with DOI and headshakes are recorded 10 minutes after DOI injection for 10 minutes. The headshake response is a rapid, rhythmic shaking of the head in a radial motion.

1. A compound of Formula I: 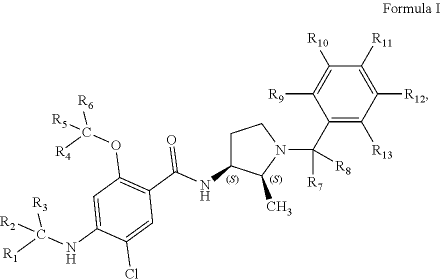
wherein:
R₁, R₂, R₃, R₄, R₅, R₆, R₇, R₈, R₉, R₁₀, R₁₁, R₁₂, and R₁₃ are independently selected from H and D; and wherein
at least one of R₁, R₂, and R₃, is D;
and at least one of R₉, R₁₀, R₁₁, R₁₂, and R₁₃ is D;
in free or salt form.

2. The compound according to claim 1, wherein the compound is in free form.

3. The compound according to claim 1, wherein the compound is in pharmaceutically acceptable salt form.

4. The compound according to claim 1, wherein each of R₁, R₂, and R₃ are D.

5. The compound according to claim 1, wherein each of R₄, R₅, and R₆ are D.

6. The compound according to claim 1, wherein R₇ and R₈ are D.

7. The compound according to claim 1, wherein each of R₉, R₁₀, R₁₁, R₁₂, and R₁₃ are D.

8. (canceled)

9. A compound, wherein the compound is: 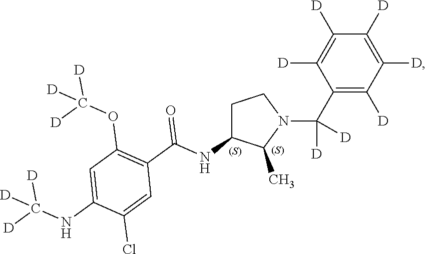
in free or salt form.

10. The compound according to claim 1, wherein the compound, in free or pharmaceutically acceptable salt form, has greater than 90% incorporation of deuterium at one or more positions designated as deuterium.

11. A pharmaceutical composition, wherein the pharmaceutical composition comprises the compound according to claim 1, in free or pharmaceutically acceptable salt form, and a pharmaceutically acceptable carrier.

12. (canceled)

13. A method for treatment of an affective disorder or an anxiety disorder in a patient in need thereof, wherein the method comprises administering to the patient the compound according to claim 1, in free or pharmaceutically acceptable salt form.

14. A method for treatment of depression, an anxiety disorder, psychosis, schizophrenia, schizoaffective disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder, or a migraine in a patient in need thereof, wherein the method comprises administering to the patient the compound according to claim 1, in free or pharmaceutically acceptable salt form.

15. The method according to claim 13, wherein the anxiety disorder is panic disorder, social anxiety disorder, a phobia, or generalized anxiety disorder.

16. A method for treatment of anhedonia, depression associated with anhedonia, suicidal ideation, anxious depression, inflammatory depression, treatment-resistant depression, dysthymia, bipolar depression, psychotic depression, or post-psychotic depression in a patient in need thereof, wherein the method comprises administering to the patient the compound according to claim 1, in free or pharmaceutically acceptable salt form.

17. The method according to claim 6, wherein the method is for treatment of depression associated with anhedonia.

18. The method according to claim 6, wherein the method is for treatment of anhedonia.

19. The method according to claim 14, wherein the method is for treatment of melancholic depression.

20. The method according to claim 14, wherein the method is for treatment of major depressive disorder.

21. A method for treatment of substance use disorder in a patient in need thereof, wherein the method comprises administering to the patient the compound according to claim 1, in free or pharmaceutically acceptable salt form.

22. (canceled)

23. (canceled)

24. (canceled)
